(19) United States
(12) Patent Application Publication
(10) Pub. No.: US 2006/0100634 A1
(43) Pub. Date: May 11, 2006

(54) Technique and instrumentation for measuring and preparing a vertebral body for device implantation using datum block

(75) Inventor: Joe Ferguson, Collierville, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 10/984,490

(22) Filed: Nov. 9, 2004

(51) Int. Cl. A61B 17/58

(52) U.S. Cl. 606/90

(57) ABSTRACT

A vertebral endplate preparation assembly is disclosed for preparing an endplate of a single vertebral body in a vertebral column to receive an implant. The assembly comprises a datum block for connecting to the single vertebral body, measuring instruments, and a cutting guide attached to the datum block. A cutting instrument is used for preparing the endplate, and an instrument coupling assembly is connected between the cutting instrument and the cutting guide.

### BACKGROUND

Recently, technical advances in the design of joint reconstructive devices have revolutionized the treatment of degenerative joint disease, moving the standard of care from arthrodesis to arthroplasty. Reconstruction of a damaged joint with a functional joint prosthesis to provide motion and to reduce deterioration of the adjacent bone and adjacent joints is a desirable treatment option for many patients. For the surgeon performing the joint reconstruction, specialized instrumentation and surgical methods may be useful to facilitate precise placement of the prosthesis.

### SUMMARY

In one embodiment, a vertebral endplate preparation assembly is disclosed for preparing an endplate of a single vertebral body in a vertebral column to receive an implant. The assembly comprises a datum block for connecting to the single vertebral body. From datum block, a measuring instrument or cutting guide may be attached. A cutting instrument is used for preparing the endplate, and an instrument coupling assembly is connected between the cutting instrument and the cutting guide.

In another embodiment, a datum block is disclosed for attachment to a single vertebral body in a vertebral column. The datum block comprises a bottom surface shaped to conform to an outer surface of the vertebral body and a channel portion shaped to interlock with a bone measuring and preparation fixture. The datum block further comprises a tool connection portion for positioning a distraction tool and an aperture in the bottom surface adapted for inserting a bone fastener into the vertebral body.

In still another embodiment, a distraction assembly is disclosed for separating a pair of vertebral bodies. The assembly comprises a first handle assembly pivotally connected to a second handle assembly, a first terminal member pivotally connected to the first handle, and a second terminal member pivotally connected to the second handle. The first and second terminals maintain a parallel distraction between the pair of vertebral bodies as the first handle assembly is moved relative to the second handle assembly.

In still another embodiment, a method of preparing a first vertebral endplate to receive an implant comprises attaching a first datum block to a first vertebral body, attaching a measuring instrument to check the size of the vertebral body, and attaching a cutting guide to the first datum block. The cutting guide including first and second rotary guides between which an alignment bar extends. The method further comprises attaching an instrument coupling assembly to the cutting guide and attaching a cutting instrument, having a cutting head, to the instrument coupling assembly. The first vertebral endplate is shaped to receive the implant.

### BRIEF DESCRIPTION OF THE DRAWINGS

FIG. 1 is a side view of a vertebral column having a damaged disc.

FIG. 2 is a flowchart describing a surgical technique.

FIG. 3 is an isometric view of an alignment guide according to an embodiment of the current disclosure.

FIG. 4 is a perspective view of a distractor assembly according to a one embodiment of the current disclosure.

FIG. 5 is a perspective view of a portion of the distractor assembly of FIG. 4.

FIG. 6 is an environmental view of the distractor assembly of FIG. 4.

FIG. 7 is a perspective view of a cutting assembly according to one embodiment of the current disclosure.

FIGS. 8-9 are perspective views of an instrument guide according to one embodiment of the current disclosure.

FIG. 10 is a perspective view of an instrument coupling assembly according to one embodiment of the current disclosure.

FIG. 11 is an environmental view of the cutting assembly of FIG. 7, the instrument guide of FIGS. 8-9, and the instrument coupling assembly of FIG. 10.

FIG. 12 is an environmental view of the cutting assembly of FIG. 7, the instrument guide of FIGS. 8-9, the instrument coupling assembly of FIG. 10 and the distractor assembly of FIG. 4.

FIGS. 13a is an environmental view of the cutting assembly of FIG. 7, the instrument guide of FIGS. 8-9, and the instrument coupling assembly of FIG. 10 in a first cutting position.

FIGS. 13b is an environmental view of the cutting assembly of FIG. 7, the instrument guide of FIGS. 8-9, and the instrument coupling assembly of FIG. 10 in a second cutting position.

FIG. 14 is an instrument coupling assembly according to another embodiment of the current disclosure.

FIG. 15 is a distractor assembly according to another embodiment of the current disclosure.

### DETAILED DESCRIPTION

The present disclosure relates generally to the field of orthopedic surgery, and more particularly to instrumentation and methods for vertebral reconstruction. For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the invention relates.

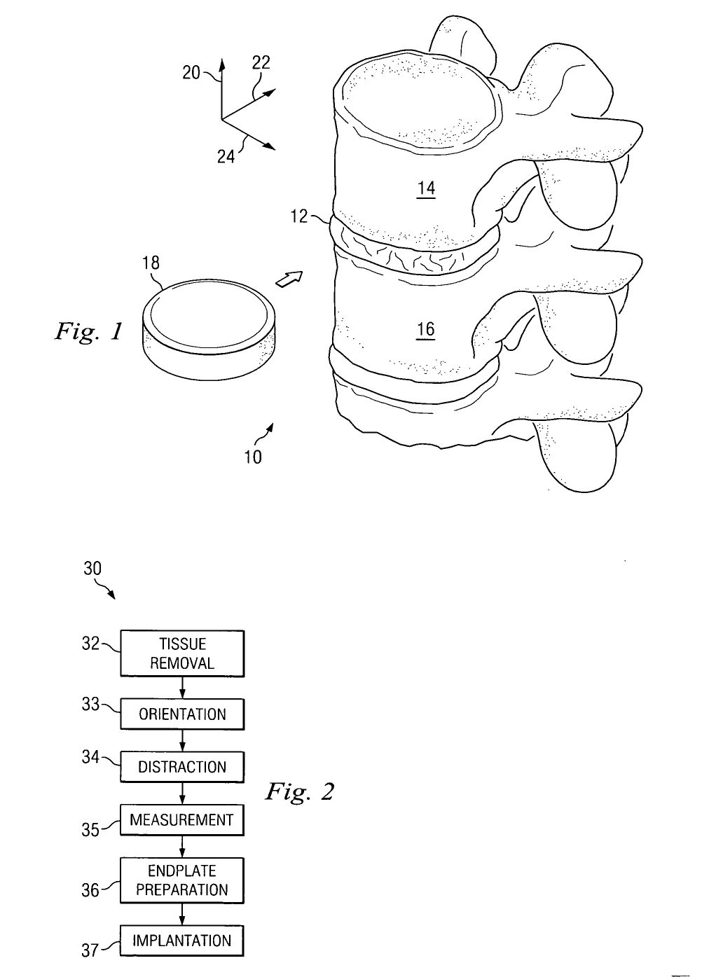

Referring first to FIG. 1, the numeral 10 refers to a vertebral column having a joint location which in this example includes an injured, diseased, or otherwise damaged intervertebral disc 12 extending between vertebrae 14, 16. The damaged disc may be replaced by an intervertebral disc prosthesis 18 which may be any of a variety of devices including the prostheses which have been described in U.S. Pat. Nos. 5,674,296; 5,865,846; 6,156,067; 6,001,130 and in U.S. Patent Application Nos. 2002/0128715 and 2003/0135277 which are incorporated by reference herein. A longitudinal axis 20 may be generally defined by the vertebral column 10. A sagittal axis 22 may extend in an anterior posterior direction, and a lateral axis 24 may extend in a transverse direction.

A surgical technique for repairing the damaged joint may be represented, in one embodiment, by the flowchart 30 depicted in FIG. 2. Referring first to step 32, all or a portion of the damaged disc 12 may be excised. This procedure may be performed using an anterior, anterolateral, lateral, or other approach known to one skilled in the art, however, the following embodiments will be directed toward a generally anterior approach. Generally, the tissue removal procedure 32 may include positioning and stabilizing the patient. Fluoroscopic or other imaging methods may be used to assist with vertebral alignment and surgical guidance. Imaging techniques may also be used to determine the proper sizing of the intervertebral prosthesis 18. In one embodiment, a sizing template may be used to pre-operatively determine the correct prosthesis size. The tissue surrounding the disc space may be retracted to access and verify the target disc space. Next, the area of the target disc may be prepared by removing excess bone, including osteophytes which may have developed, and other tissues which may include portions of the annulus and all or portions of the nucleus pulpous. The tissue removal procedure 32, which may include a discectomy procedure, may alternatively or additionally be performed after alignment and/or measurement procedures have been taken.

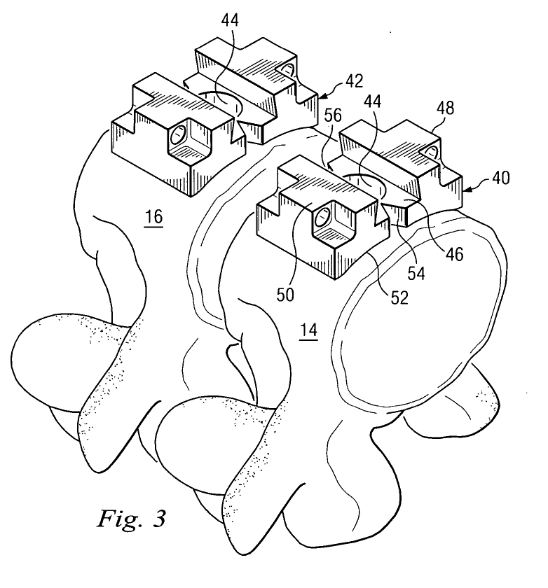

Proceeding to step 33 of the surgical technique 30 of FIG. 2, various orientation and location procedures may be conducted in preparation for implantation of the disc prosthesis 18. The transverse center of the disc space may be determined and marked. Referring now to FIG. 3, a pair of datum blocks 40, 42 may be attached directly to the surfaces of the vertebral bodies 14, 16, respectively. The datum block 42 may be substantially similar to datum block 40 and therefore will not be described in detail. Datum block 40 may include a vertebral body attachment aperture 44, an attachment guide 46, and tool guides 48, 50. In this embodiment, the attachment guide 46 may be a dove tailed groove, but it is understood that in alternative embodiments, the attachment guide may be either the male or female component of an interlocking assembly such as a dovetailed or T-shaped coupling. The datum block 40 may include a base portion 52 which may be saddle-shaped. The datum block 40 may further include alignment guides 54, 56. The datum blocks 40, 42 may be relatively low profile and allow for improved visibility of the surgical site.

During the orientation procedures of step 2, the datum block 40 may be centered on the vertebral body 14 by aligning the alignment guides 54, 56 with the transverse centering mark. The block 40 is secured to the vertebral body 14 by a fastener, such as a screw (not shown), installed through the attachment aperture 44. The datum block 40 uses the external anatomy of the individual vertebral body 14 to set up proper location and orientation. The datum block 40 may be used for attaching and/or aligning instrumentation used for distraction, measuring, bone preparation, or prosthesis insertion. Block 42 may be located on vertebral body 16 in substantially the same way as described above for block 40. With the datum blocks 40, 42 attached as disclosed above, the blocks may independently follow the vertebral bodies 14, 16.

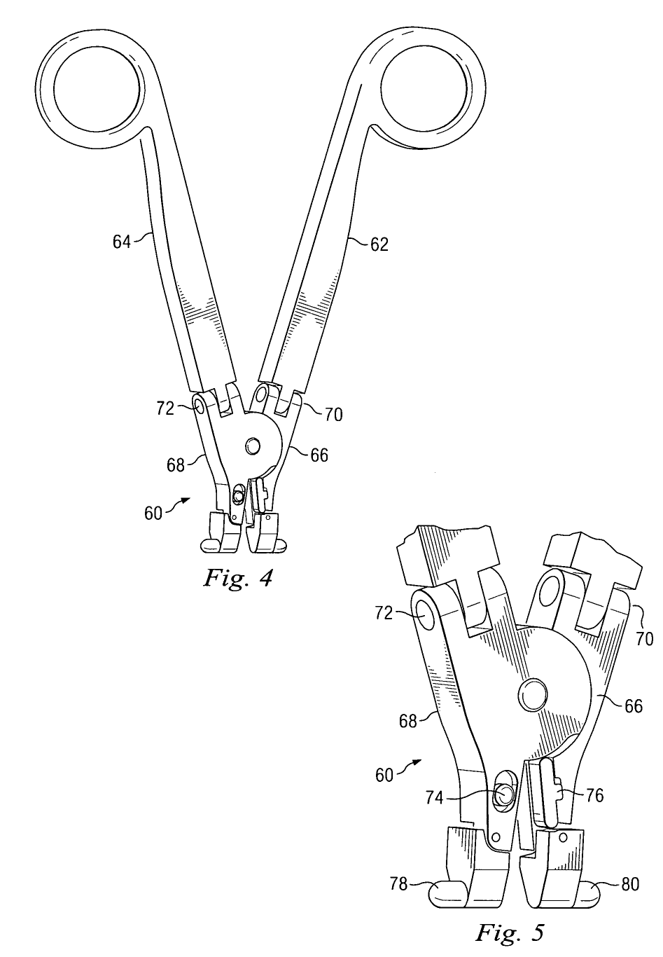

Proceeding to step 34 of the surgical technique 30 of FIG. 2, a spreader or distractor assembly 60, as shown in FIGS. 4-6, may be introduced. The distractor assembly 60 may include handles or arms 62, 64 connected to cam mechanisms 66, 68 by handle joints 70, 72, respectively. Cam mechanisms 66, 68 may engage cam sliders 74, 76, respectively, which may in turn, moveably engage terminals 78, 80, respectively. Cam mechanism 66 may be rotatably coupled to cam mechanism 68, and terminals 78, 80 may be pivotally coupled to cam mechanisms 68, 66, respectively.

In operation, the terminal 78 may engage the tool guide 48, and the terminal 80 may engage a corresponding tool guide on the datum block 42. With the distractor assembly 60 engaged, the vertebral bodies 14, 16 may be distracted by drawing the arms 62, 64 together. As the arms 62, 64 are drawn together, the cam mechanisms 66, 68 may engage the cam sliders 74, 76, respectively, which may in turn move the terminals 78, 80, respectively. As the terminals 78, 80 move apart, a relatively parallel displacement may be maintained between the terminals and correspondingly, between the vertebral bodies 14, 16. The vertebral bodies 14, 16 may be placed in tension, providing access to the intervertebral space to allow further discectomy and/or decompression procedures as needed. The arms 62, 64 may bend at the handle joints 70, 72 to open the operating field. The arms 62, 64 may also be locked in the distracted position to maintain the operating field.

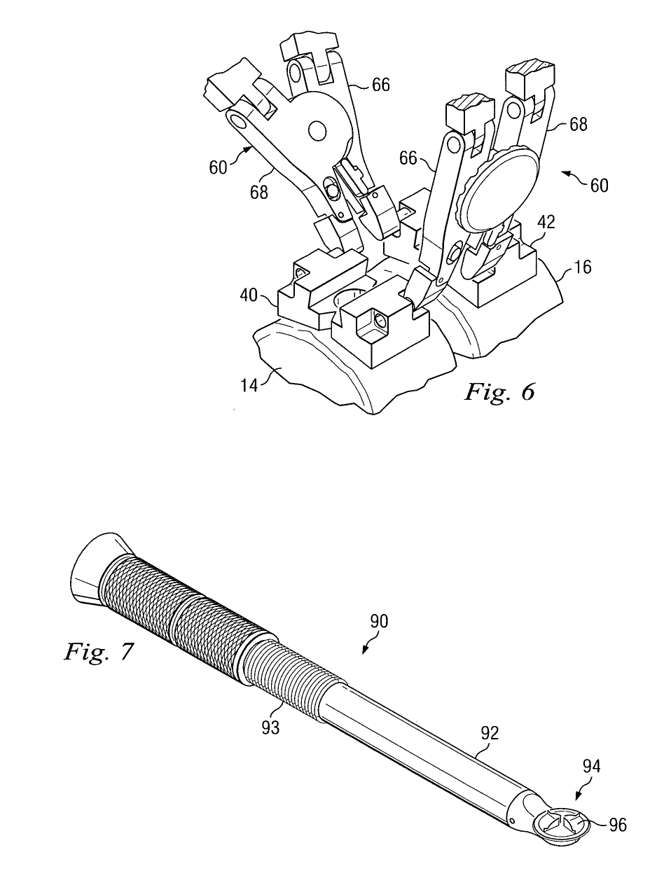

Although the use of only one distractor assembly 60 has been described, it is understood that a second distractor, as shown in FIG. 6, may be used. In an alternative embodiment, a similar distractor assembly may have a scissor-style configuration, such that as the arms are drawn apart, the terminals also are drawn apart.

Referring again to FIG. 2, with the datum blocks 40, 42 attached to the distracted vertebral bodies 14, 16, the surgical technique 30 may then proceed to step 35. At step 35, measurements, such as a depth measurement, may be performed at the disc site to determine the proper sizing of instrumentation and devices to be used throughout the remainder of the surgical technique 30. Measuring the intervertebral space may involve the use of a variety of instrumentation and equipment including, for example, the measurement instrumentation described in U.S. patent application Ser. No. 10/799,835 which is incorporated by reference herein.

Referring again to FIG. 2, the surgical technique 30 may proceed to step 36 for further preparation of the vertebral endplate surfaces. Referring now to FIG. 7, to prepare the endplate surfaces to provide a secure seat for the intervertebral prosthesis 18, a milling or cutting instrument 90 may be provided. In the embodiment of FIG. 7, the cutting instrument 90 may comprise a shaft 92 and a cutting head 94 having a cutting surface 96. A portion of the shaft 92 may include threads 93.

The cutting instrument described above for FIG. 7 is merely one embodiment which may be used with the distractor assembly 60 and the anchoring devices 40, 42. In alternative embodiments, the cutting instrument may include a burr or other cutting surfaces known in the art. The cutting instrument may also include a telescoping shaft to permit lengthening of the cutting instrument. The cutting instrument 90 may be substantially similar to one of the cutting instrument embodiments described in the above referenced U.S. patent application Ser. No. 10/799,835.

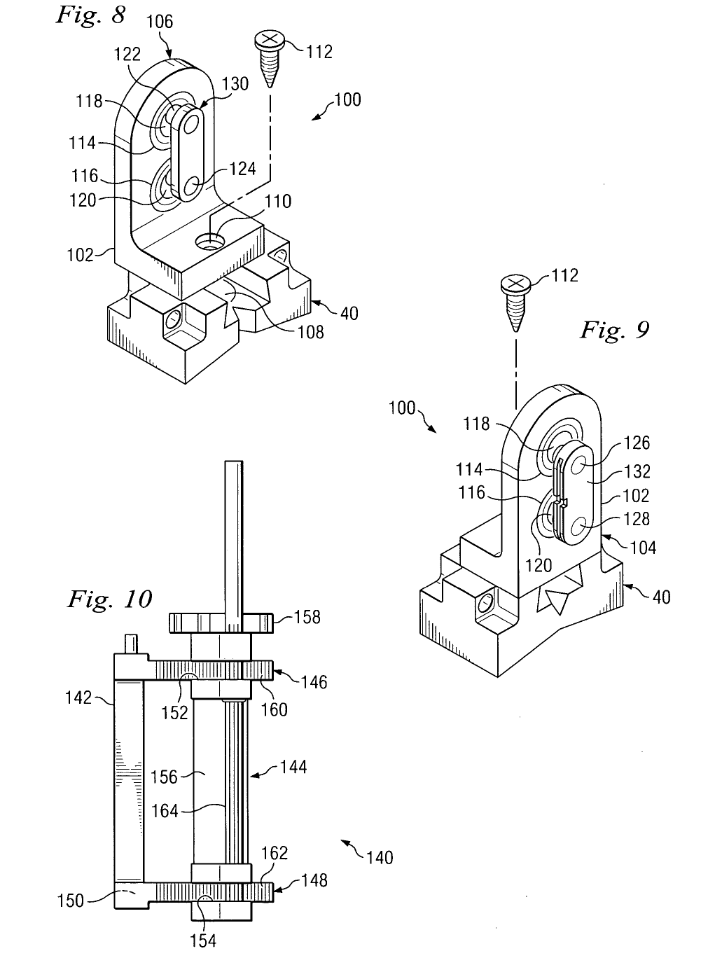

Referring now to FIGS. 8 and 9, a cutting guide 100 may also be used to prepare the vertebral endplate surfaces. The cutting guide 100 may include a body 102 having a tool interface side 104, an external side 106, and a connection portion 108 for interlocking with the attachment guide 46 of the datum block 40. In this embodiment, the connection portion 108 is a dove tail shaped projection, but other interlocking mechanisms are also suitable. The cutting guide 100 may also include an aperture 110 through which an interlock fastener 112 may extend to secure the interface between the cutting guide 100 and the datum block 40. The cutting guide 100 may house a set of sealed bearings 114, 116 which permit movement of a set of rotary guides 118, 120. A set of cam spindles 122, 124 may extend, offset from the center, from the rotary guides 118, 120, respectively, on the external side 106 of the cutting guide 100. A set of cam spindles 126, 128 may extend, offset from the center, from the rotary guides 118, 120, respectively, on the tool interface side 104 of the cutting guide 100. An alignment bar 130 may extend between the cam spindles 122, 124 on the external side 106 of the cutting guide 100, and a coupling bar 132 may extend between the cam spindles 126, 128 on the tool interface side 104.

Referring now to FIG. 10, an instrument coupling assembly 140 for connecting the cutting guide 100 to the cutting instrument 90 may include an attachment device 142 and a tool positioning device 144. The attachment device 142 may include forked arms 146, 148 and a channel 150 for locking to the coupling bar 132. The tool positioning device 144 may include channels 152, 154 configured to mate with and slide along the forked arms 146, 148, respectively. The tool positioning device 144 may also include a tubular sleeve 156 through which the cutting instrument 90 may extend. The tool positioning device 144 may also include an adjustment dial 158 for adjusting the position of the cutting instrument 90 relative to the tool positioning device 144. The adjustment dial 158 may be threadedly engaged with the cutting instrument 90.

The forked arms 146, 148 of the attachment device 142 may include toothed surfaces 160, 162 configured to engage a pinion gear 164 extending from the tool positioning device 144. This rack and pinion system formed by the toothed surfaces 160, 162 and gear 164 allow the tool positioning device 144 to move along and lock to the forked arms 146, 148. Connections between the components of the cutting guide 100 and instrument coupling assembly 140 may be secured, as needed, with fasteners such as pins and screws.

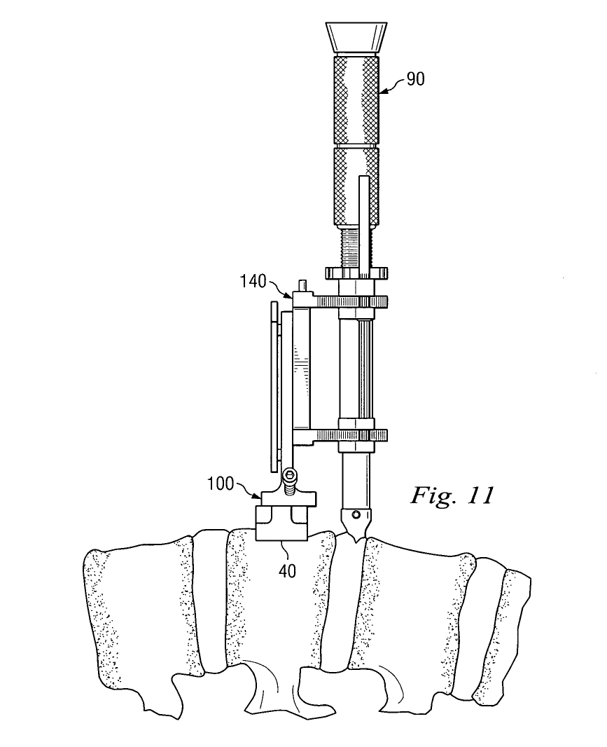

Referring now to FIGS. 11, 12, 13a, and 13b, based upon the measurements taken in step 35 and the size and profile of the prosthesis 18 to be implanted, the cutting surface 96 may be selected. The cutting instrument 90 with the selected cutting surface 96 may be assembled to the tool positioning device 144 as described above. With the datum block 40 attached to the vertebral body 14, the cutting guide 100 may be mounted to the datum block 40 as described above. The attachment device 142 may be mounted to cutting guide 100 as described above, and the tool positioning device 144 coupled with the cutting instrument 90 may be mounted to the attachment device 142 also as described above.

With the attachment device 142 and the tool positioning device 144 interconnected by the gear 164 and the toothed surfaces 160, 162, the depth of the cut made by the cutting head 94 along the longitudinal axis 20 may be adjusted by rotating the pinion gear 164. Using the adjustment dial 158, the anterior-posterior placement of the cutting head 94 along the sagittal axis 22 may be adjusted. The proper positioning of the cutting head 94 may be established with known offsets and may be verified with fluoroscopic or other imaging techniques.

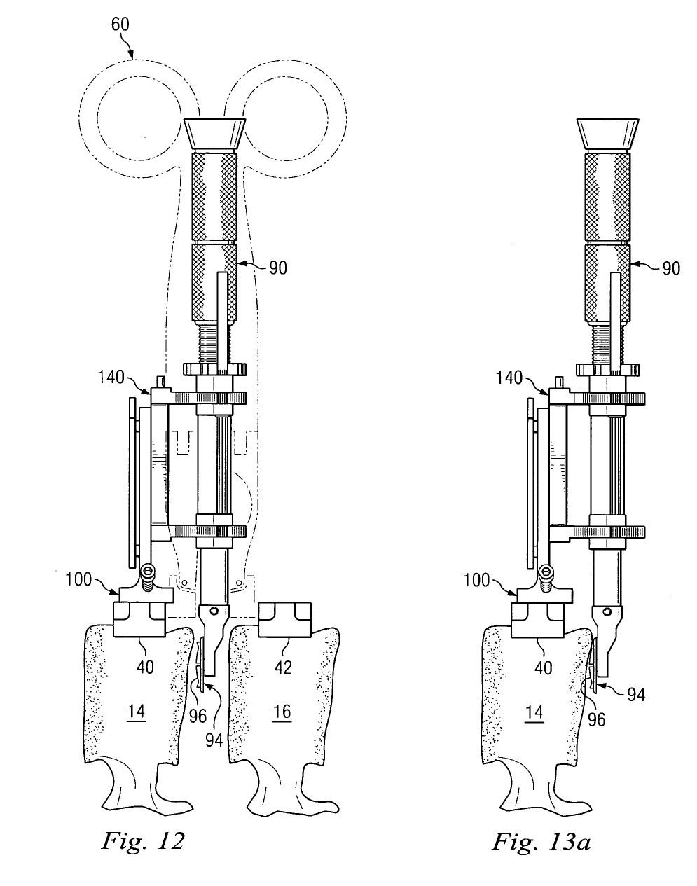

As shown in FIG. 12, the distractor assembly 60 may remain in place during the cutting procedures. The arms of the distractor assembly 60 may break away from the surgical site to provide more space to the surgeon. If desired, more than one distractor assembly may be used to maintain the disc space.

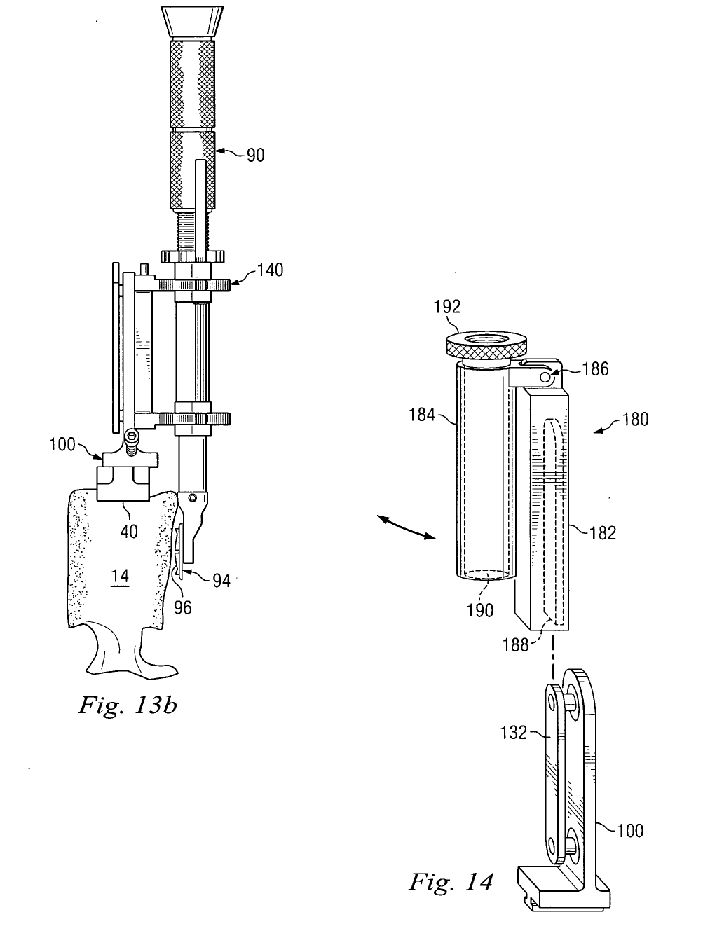

In operation, a user may cause the cutting instrument 90 to travel a relatively circular path predetermined by the relationship between the centers of the rotary guides 118, 120 and the location of the cam spindles 126, 128, respectively. As shown in FIG. 13a, when the cam spindles 126, 128 are rotated to a position directly above the center of the rotary guides, 118, 120, respectively, the cutting head 94 may be in an uppermost position in its circular path. As shown in FIG. 13b, when the cam spindles 126, 128 are rotated to a position directly below the center of the rotary guides, 118, 120, respectively, the cutting head 94 may be in a lowermost position in its circular path. As the cutting instrument 90 travels its path, the cutting surface 96 may be powered to cut, mill, or otherwise shape the vertebral body 14. The rotary guides 118, 120 may be rotated by driving shaft 92 of the cutting instrument 90, driving the alignment bar 130, driving one or both of the cam spindles 118, 120, directly, or any other method of driving the cutting head 94 through its predetermined path as may be appreciated by one skilled in the art. The path of the cutting head 94 may be adjusted by adjusting the adjustment dial 158 to raise or lower the cutting instrument 90 relative to the vertebral bodies 14, 16.

The cutting surface 96 may be shaped such that the profile that it creates in the vertebral endplate matches the profile of the selected intervertebral prosthesis 18 to create a secure seat for the prosthesis. After the first endplate is prepared, the cutting instrument 90 may be mounted to the datum block 42 with the cutting surface 96 positioned adjacent to the endplate of the vertebral body 16. The cutting instrument 90 may again be powered, this time to shape the endplate of vertebral body 16. If multi-level surgical procedures, involving more than one intervertebral disc location, are required, the use of datum block allows bridging across several disc spaces without removing and resetting instrumentation. As described above, the datum blocks 40, 42 may be independently fixed and aligned with their respective vertebral bodies. As such, the vertebral bodies 14, 16 may be permitted to move independently of each other and therefore, the endplate preparation procedure may permit each of the vertebral bodies to be shaped independently. In one embodiment, the datum blocks 40, 42 may move independently in any direction so as to permit the best access and apply the least amount of stress to the system. In an alternative embodiment, the datum blocks 40, 42 may permit independent movement of the vertebral bodies 14, 16 in the sagittal plane while maintaining alignment of the vertebral bodies 14, 16 in the transverse and coronal planes.

Referring again to FIG. 2 at step 37, after the vertebral endplates are prepared, the cutting instrument 90, the cutting guide 100, and the instrument coupling assembly 140 may be removed from the datum block 40 in preparation for implanting the intervertebral prosthesis 18. With the cutting instrumentation removed, the intervertebral prosthesis 18 may be inserted into the prepared space using any of a variety of insertion methods. In some embodiments, the datum blocks 40, 42 may be used to guide prosthesis insertion instrumentation. After the prosthesis 18 is implanted, the tension on the distractor assembly 60 may be released. The datum blocks 40, 42 may be removed form the vertebral bodies 14, 16 respectively. With all instrumentation removed from the disc site, the wound may be closed.

Referring now to FIG. 14, in an alternative embodiment, an instrument coupling assembly 180 for connecting the cutting guide 100 to the cutting instrument 90 may include an attachment device 182 and a tool positioning device 184 connected by a hinge joint 186. The attachment device 182 may include a channel 188 for locking to the coupling bar 132. The tool positioning device 184 may include a tubular sleeve 190 through which the cutting instrument 90 may extend. The tool positioning device 184 may also include an adjustment dial 192 for adjusting the position of the cutting instrument 90 relative to the tool positioning device 184. The adjustment dial 192 may be threadedly engaged with the cutting instrument 90. The hinge joint 186 may pivot to allow angular displacement between the cutting instrument 90 and the attachment device 182.

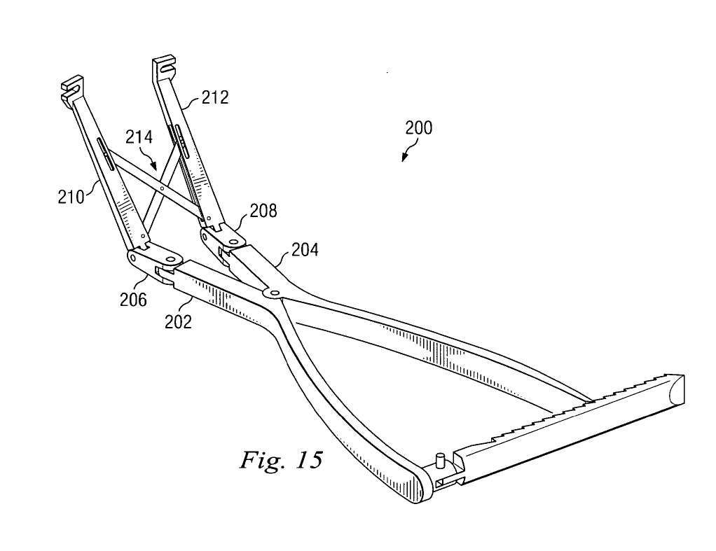

Referring now to FIG. 15, in an alternative embodiment a distractor assembly 200 may be used to distract the vertebral bodies 14, 16 in parallel alignment. The distractor assembly 200 may include pivotally connected handles 202, 204. The handles 202, 204 may be connected by hinges 206, 208 to legs 210, 212, respectively. The legs 210, 212 may be slideably connected to a cross-bar mechanism 214. The distractor assembly 200 may perform substantially the same type of vertebral body distraction as described above for distractor assembly 60.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims. It is understood that one skilled in the art may omit or add minor steps to the described procedures and that such expanded or abbreviated methods are intended to be included within the scope of this invention. In the claims, means-plus-function clauses are intended to cover the structures described herein as performing the recited function and not only structural equivalents, but also equivalent structures.

What is claimed is:

1. A datum block for attachment to a single vertebral body in a vertebral column, the datum block comprising:
a bottom surface shaped to conform to an outer surface of the vertebral body;
a channel portion shaped to interlock with a bone preparation fixture;
a tool connection portion for positioning a distraction tool; and
an aperture in the bottom surface adapted for inserting a bone fastener into the vertebral body.

2. The datum block of claim 1 wherein the channel portion is dove-tail shaped

3. The datum block of claim 1 wherein the bottom surface is saddle shaped.

4. The datum block of claim 1 wherein the bottom surface is adapted for self centering on the vertebral body.

5. The datum block of claim 1 further comprising a centering indicator for aligning the datum block along a transverse center.

6. A distraction assembly for separating a pair of vertebral bodies, the assembly comprising:
a first handle assembly pivotally connected to a second handle assembly;
a first terminal member pivotally connected to the first handle; and
a second terminal member pivotally connected to the second handle;
wherein the first and second terminals create a distraction between the pair of vertebral bodies as the first handle assembly is moved relative to the second handle.

7. The distraction assembly of claim 6 wherein the first and second terminals create a parallel distraction between the pair of vertebral bodies as the first handle assembly is moved relative to the second handle assembly.

8. The distraction assembly of claim 6 wherein the first and second terminals create a free rotational distraction between the pair of vertebral bodies as the first handle assembly is moved relative to the second handle assembly.

9. The distraction assembly of claim 6 wherein
the first handle assembly includes a first cam mechanism;
the second handle assembly includes a second cam mechanism;
and wherein the first cam mechanism is rotatably connected to the second cam mechanism.

10. The distraction assembly of claim 9 further comprising:
a first cam slider movable between the second cam mechanism and the first terminal member and
a second cam slider movable between the first cam mechanism and the second terminal member.

11. The distraction assembly of claim 6 wherein
the first handle assembly includes a first joint and a first grip portion,
wherein the first grip portion is bendable toward the first terminal portion at the first joint.

12. The distraction assembly of claim 6 further comprising:
a locking mechanism for locking the position of the first handle assembly with respect to the second handle assembly.

13. A vertebral endplate preparation assembly for preparing an endplate of a single vertebral body in a vertebral column to receive an implant, the assembly comprising:
a datum block for connecting to the single vertebral body;
a cutting guide attached to the datum block;
a cutting instrument for preparing the endplate; and
an instrument coupling assembly connected between the cutting instrument and the cutting guide.

14. The assembly of claim 13 wherein the cutting guide comprises at least two rotary guides.

15. The assembly of claim 14 wherein the at least two rotary guides each move on a set of sealed bearings.

16. The assembly of claim 14 further comprising a coupling bar extending between the at least two rotary guides.

17. The assembly of claim 16 wherein each rotary guide includes a cam spindle and the coupling bar extends between the cam spindles.

18. The assembly of claim 13 further comprising an adjustment apparatus for moving the cutting instrument along an anterior posterior axis.

19. The assembly of claim 18 wherein the adjustment apparatus comprises a dial on the instrument coupling assembly threadedly engaged with the cutting instrument.

20. The assembly of claim 13 wherein the instrument coupling assembly comprises an adjustment apparatus for moving the cutting instrument along a generally logitudinal axis defined by the vertebral column.

21. The assembly of claim 20 wherein the adjustment apparatus comprises a rack and pinion assembly.

22. The assembly of claim 13 wherein the instrument coupling assembly comprises at least one pair of forked arms.

23. The assembly of claim 13 further comprising a distractor for separating at least two vertebral bodies in the vertebral column.

24. A method of preparing a first vertebral endplate to receive an implant, the assembly comprising:
attaching a first datum block to a first vertebral body;
attaching a cutting guide to the first datum block, the cutting guide including first and second rotary guides between which an alignment bar extends;
attaching an instrument coupling assembly to the cutting guide;
attaching a cutting instrument, having a cutting head, to the instrument coupling assembly; and
shaping the first vertebral endplate to receive the implant.

25. The method of claim 24 further comprising:
adjusting the longitudinal position of the cutting instrument relative to the first datum block.

26. The method of claim 24 further comprising:
adjusting the position of the cutting instrument relative to the first datum block along a sagittal axis.

27. The method of claim 24 further comprising:
rotating the first and second rotary guides in unison, while powering the cutting instrument.

28. The method of claim 24 further comprising:
driving the alignment bar to move the cutting head in a predetermined path.

29. The method of claim 24 further comprising:
driving the cutting instrument to move the cutting head in a predetermined path.

30. The method of claim 24 further comprising:
attaching a second datum block to a second vertebral body;
removing the cutting guide from the first datum block and attaching the cutting guide to the second datum block; and
shaping a second vertebral endplate to receive the implant.

31. The method of claim 30 further comprising:
attaching a distractor assembly, having a pair of terminal portions, between the first and the second datum blocks; and
separating the first and second vertebral bodies while maintaining a parallel alignment between the terminal portions.

32. The method of claim 30 further comprising:
moving the first datum block independently of the second datum block.
